(19) United States
(12) Patent Application Publication
(10) Pub. No.: US 2007/0060763 A1
(43) Pub. Date: Mar. 15, 2007

(54) PROCESS FOR WORKING UP FATTY COMPOUNDS

(76) Inventors: Stefan Franzen, Kamen (DE); Thomas Kapala, Ratingen (DE); Eike Ulf Mahnke, Velbert (DE); Yiwen Dong, Dortmund (DE)

Correspondence Address: COGNIS CORPORATION;PATENT DEPARTMENT, 300 BROOKSIDE AVENUE, AMBLER, PA, 19002, US

(21) Appl. No.: 11/463,396

(22) Filed: Aug. 9, 2006

(30) Foreign Application Priority Data
Aug. 10, 2005 (DE) 10 2005 037 707.6

(51) Int. Cl. C07C 51/43

(52) U.S. Cl. 554/174

(57) ABSTRACT

The invention relates to a process for working up fatty compounds from esterification and transesterification reactions, wherein, the fatty compounds are contacted with ion exchangers to remove unwanted constituents.

### FIELD OF THE INVENTION

This invention relates generally to the field of oleochemistry and, more particularly, to a new process for working up fatty compounds from esterification and transesterification reactions, more particularly for refining and deacidifying such compounds.

### BACKGROUND OF THE INVENTION

In the refining of fatty compounds from esterification and transesterification reactions, free acids are removed by chemical or physical methods to increase stability in storage and product quality. The most simple method is to saponify the free acids by addition of alkali. In this connection, a distinction is drawn between dry and wet refining.

Dry refining uses solid or bound sodium hydroxide. In this process, soaps may be formed as neutralization products. By contrast, aqueous sodium hydroxide or soda solution and a large amount of water are added in wet refining, so that the water then has to be removed. In addition, soap formed has to be removed from the reaction mixture by repeated washing. The reaction product is then optionally subjected to deodorizing, drying and filtration steps.

Known processes are attended by the disadvantage that, because the acids are quantitatively converted into soaps, elaborate, time- and energy-consuming purification steps, more particularly washing and filtrations, have to be carried out. At the same time, large amounts of wastewater accumulate and, again, have to be laboriously purified or expensively disposed of. After the refining process, the free acids in the products from the esterification or transesterification are finally present as soaps.

Accordingly, the complex problem addressed by the present invention was to provide an improved process for working up products from esterification and transesterification reactions for refining and deacidification which would be distinguished from the prior art by a significant reduction in the number of purification steps and in the volume of wastewater. At the same time, losses through product saponification would also be reduced. Finally, the process according to the invention would also be workable in such a way that catalyst impurities, such as sulfate anions and mono- or polyvalent metal cations for example, could also be removed.

### BRIEF DESCRIPTION OF THE INVENTION

The present invention relates to a process for working up fatty compounds from esterification and transesterification reactions which is characterized in that the fatty compounds are treated with ion exchangers.

The advantage of the process according to the invention is, in particular, that, through the use of anion exchangers, the free acid is directly bound to the ion exchanger. Water is only formed in stoichiometric quantities and is subsequently removed in the drying step. In particular, there is no need for the laborious removal of soap by washing or for filtration and, at the same time, the quantity of wastewater is significantly reduced. By virtue of the lower basicity of the anion exchanger and the milder reaction temperatures, there are also fewer losses through product saponification than in traditional wet refining. At the same time, other troublesome anions, particularly the unwanted sulfates, are also removed. In addition, by carrying out cation and anion exchanging steps in line, monovalent or polyvalent metal ions can also be removed. As in water-based ion exchanger systems, the ion exchangers can be regenerated with dilute aqueous sodium or potassium hydroxide and dilute hydrochloric or sulfuric acid.

### DETAILED DESCRIPTION OF THE INVENTION

Brief Description of the Drawings:

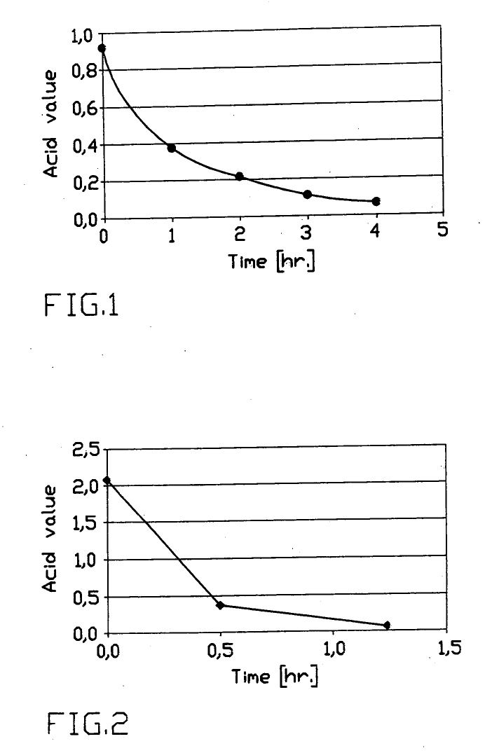

FIG. 1 Shows a plot of acid value vs. time for Example 1.

FIG. 2 Shows a plot of acid value vs. time for Example 2.

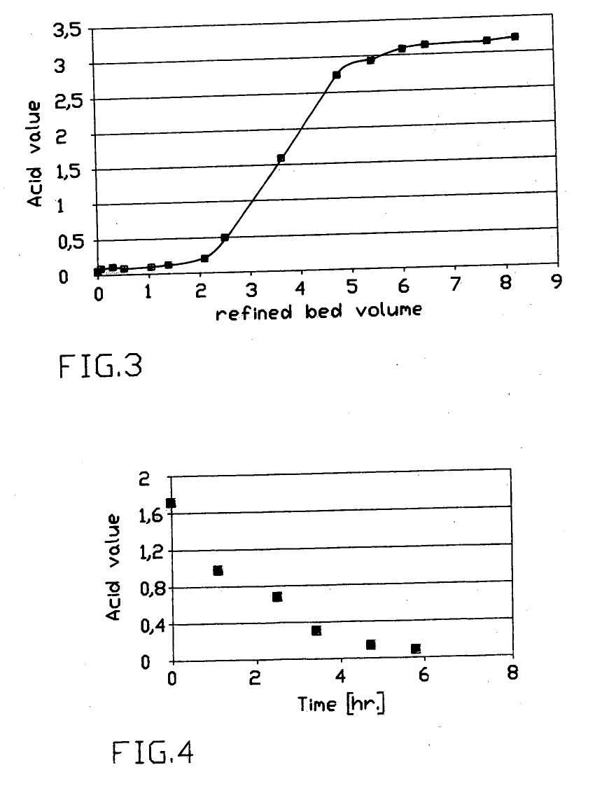

FIG. 3 Shows a plot of acid value vs. bed volume passed through static bed of Example 3.

FIG. 4 Shows a plot of acid value vs. time for Example 4

### STARTING MATERIALS

The choice of the esterification or transesterification products is not critical to the process according to the invention.

In a first and preferred embodiment of the process according to the invention, glycerides corresponding to formula (I) may be worked up as fatty compounds. 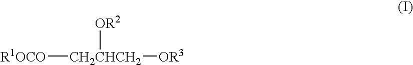
In formula (I), R¹CO is a saturated and mono- or polyunsaturated acyl group containing 2 to 22 carbon atoms and R² and R³ independently of one another represent hydrogen and mono- or polyunsaturated acyl groups containing 2 to 22 and preferably 6 to 18 carbon atoms. Typical examples are vegetable oils, such as for example coconut oil, palm oil, palm kernel oil, olive oil, sunflower oil, thistle oil, rapeseed oil, almond oil and the like.

In an alternative embodiment, esters of monocarboxylic acids corresponding to formula (II) may also be used as fatty compounds:
R⁴COO—R⁵ (II)
In formula (II), R⁴CO is a saturated and mono- or polyunsaturated acyl group containing 2 to 22, preferably 6 to 18 and more particularly 12 to 14 carbon atoms and R⁵ is a saturated or mono- or polyunsaturated, optionally substituted alk(en)yl group containing 2 to 22, preferably 6 to 18 and more particularly 12 to 14 carbon atoms or the residue of a (poly)alkylene glycol, more particularly ethylene glycol, diethylene glycol, propylene glycol or dipropylene glycol. Typical examples are wax esters based on fatty acids containing 6 to 22 and preferably 12 to 18 carbon atoms and corresponding fatty alcohols likewise containing 6 to 22 and preferably 12 to 18 carbon atoms, such as for example myristyl myristate, myristyl palmitate, stearyl palmitate, stearyl stearate, cetearyl stearate, oleyl oleate and the like.

Finally, other suitable starting materials are esters of dicarboxylic acids corresponding to formula (III):
R⁶OOC—(A)ₙ—COOR⁷ (III)
in which A is a linear or branched alkylene or alkenylene group containing 1 to 10 carbon atoms or a phenyl group, n=0 or 1, R⁶ and R⁷ independently of one another represent saturated or mono- or polyunsaturated, optionally substituted alk(en)yl groups containing 2 to 22, preferably 6 to 22 and more particularly 12 to 18 carbon atoms or the residue of a (poly)alkylene glycol, more particularly ethylene glycol, diethylene glycol, propylene glycol or dipropylene glycol. Typical examples are the stearyl, palmityl or oleyl esters of malonic acid or adipic acid.

Overall, particular preference is attributed to synthetic or natural tri- and partial glycerides and alkyl esters which, after refining, are used in cosmetic preparations or in foods. So far as the quantity of free acids is concerned, the starting materials may have acid values in the range from 0.5 to 10.

Ion Exchangers

The use of ion exchangers for working up and catalysis in water-containing systems is known, cf. for example the softening or desalting of water, recovery from the metal. Ion exchangers are also used in chromatography. In oleochemistry, ion exchangers are used for purifying glycerol (desalting). Another application is the use of cation exchangers for acidic catalysis in esterification reactions (for example in the production of isopropyl myristate/palmitate). However, they have not yet been considered for the deacidification of esterification and transesterification products.

Anion exchangers or combinations of anion and cation exchangers arranged in series may be used in the process according to the invention, irrespective of whether only free fatty acids and optionally other troublesome anions or even metal cations are to be removed. Both ion exchangers based on polymeric resins and other solid matrices containing functional groups (for example zeolites or oxidic ion exchangers) may be used. Typical examples of anion exchangers are polymeric matrices based on polystyrene/divinylbenzene/dimethylamine, polystyrene/divinyl benzene/trimethyl ammonium chloride or polyacrylodimethylamine which are commercially available, for example, from the Purolite company. Typical examples of cation exchangers are polystyrene/divinylbenzene resins containing sulfonic acid or carboxyl groups.

Both weakly basic and strongly basic anion exchangers and strongly acidic or weakly acidic cation exchangers are suitable for use in the process according to the invention, particle sizes of 50 to 1,000 μm and preferably in the range from 300 to 800 μm having proved to be particularly advantageous. The ion exchangers may be used as temperatures of 0 to 200° according to type. Cation exchangers based on synthetic resins can usually be used at temperatures of up to at most 150° C. while anion exchangers based on synthetic resins can usually be used at temperatures of up to at most 70° C. Accordingly, working up is typically carried out at temperatures of 0 to 150° and preferably at temperatures of 25 to 95° C.

For the removal of free acids from the esterification and transesterification products, the reaction takes place on the principle of acid/base reactions. The acid anions are bound to anion exchangers, water being stoichiometrically formed:
RCOOH+RS—OH→RS—OOCR+H₂O
For removing the free acid, the anion exchangers may be used immediately after conditioning. The anion exchangers do not have to be dried or pretreated with organic substances for use. The charging capacity of the anion exchangers in the organic system is only slightly different from that in the aqueous system. Regeneration with dilute sodium hydroxide can be repeated several times.

### EXAMPLES

### Example 1

50 g of the crude product caprylic/capric acid triglyceride (Myritol® 318, Cognis Deutschland GmbH & CO. KG) were mixed with 2 g polystyrene/divinyl benzene/dimethyl amine (PFA A, Purolite) in a shaker flask and stirred continuously at room temperature. The acid value of the mixture was measured after sampling at certain time intervals (DGF-Einheitsmethode C-V 2(81)). The change in the acid value as a function of time is shown in FIG. 1. A reduction in the acid value can clearly be seen.

### Example 2

1290 g of the crude product caprylic/capric acid triglyceride (Myritol® 318, Cognis Deutschland GmbH & CO. KG) were stirred with 60 g polystyrene/divinyl benzene/trimethyl ammonium chloride (PPA-400, Purolite) at 25° C. in a stirred tank. The acid value of the mixture was measured after sampling at certain time intervals (DGF-Einheitsmethode C-V 2(81)). The change in the acid value as a function of time is shown in FIG. 2. A reduction in the acid value can clearly be seen.

### Example 3

The crude product caprylic/capric acid triglyceride (Myritol® 318, Cognis Deutschland GmbH & CO. KG) was passed at room temperature through a 3 cm diameter fixed bed packed with 42 g polystyrene/divinyl benzene/trimethyl ammonium chloride (PPA-400, Purolite). The break-through curve of the product was determined by measuring the acid value (DGF-Einheitsmethode C-V 2(81)) and is shown in FIG. 3. The linear charging rate is 1 cm/min.

### Example 4

50 g of the crude product isopropyl myristate (Cognis Deutschland GmbH & Co. KG) was mixed with 4 g polystyrene/divinyl benzene/trimethyl ammonium chloride (PFA 400, Purolite) in a shaker flask and stirred at room temperature. The crude product contains sulfuric acid as catalyst and unreacted fatty acid from the esterification reaction. The acid value of the mixture was measured after sampling at certain time intervals (DGF-Einheitsmethode C-V 2(81)). The change in the acid value as a function of time is shown in FIG. 4. A reduction in the acid value can clearly be seen.

1. A process for working up fatty compounds from esterification and transesterification reactions which comprises: contacting fatty compounds with solid ion exchangers.

2. The process as claimed in claim 1, wherein, at least one member selected from the group consisting of free acids, inorganic anions, organic anions, monovalent metal cations and polyvalent metal cations are removed from the fatty compounds.

3. The process as claimed in claim 1, wherein, the fatty compound comprises glycerides of formula: 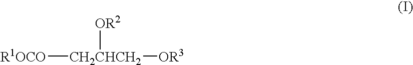
in which R¹CO is a saturated acyl group, a mono-unsaturated acyl group, or a polyunsaturated acyl group containing 2 to 22 carbon atoms and R² and R³ independently of one another represent hydrogen a mono-unsaturated acyl group or a polyunsaturated acyl group containing 2 to 22 carbon atoms.

4. The process as claimed in claim 1, wherein, the fatty compound comprises, esters of monocarboxylic acids of formula:
R⁴COO—R⁵ (II)
in which R⁴CO is a saturated acyl group, a mono-unsaturated acyl group or a polyunsaturated acyl group each containing 2 to 22 carbon atoms and R⁵ is a saturated acyl group, a mono-unsaturated acyl group, a polyunsaturated acyl group, an optionally substituted alk(en)yl group containing 2 to 22 carbon atoms or the residue of a (poly)alkylene glycol.

5. A process as claimed in claim 1, wherein, the fatty compound comprises esters of dicarboxylic acids of formula:
R⁶OOC—(A)ₙ—COOR⁷ (III)
in which A is a linear or branched alkylene group containing 1 to 10 carbon atoms, an alkenylene group containing 1 to 10 carbon atoms or a phenyl group, n=0 or 1, R⁶ and R⁷ independently of one another represent a member selected from the group consisting of saturated, optionally substituted alk(en)yl groups containing 2 to 22 carbon atoms, mono-unsaturated optionally substituted alk(en)yl groups containing 2 to 22 carbon atoms, polyunsaturated optionally substituted alk(en)yl groups containing 2 to 22 carbon atoms, or the residue of a polyalkylene glycol.

6. The process as claimed in claim 1, wherein, the ion exchangers comprise: anion exchangers or a combination of cation and anion exchangers arranged in series.

7. The process as claimed in claim 1, wherein, the ion exchangers comprise polymeric resins or other solid matrices containing functional groups.

8. The process as claimed in claim 7, wherein, the anion exchangers comprise at least one polymer matrix selected from the group consisting of polystyrene/divinylbenzene/dimethylamine, polystyrene/divinylbenzene/trimethyl ammonium chloride and polyacrylodimethylamine.

9. The process as claimed in claim 1, wherein, the contacting is carried out at temperatures of 0 to 150° C.

10. The process as claimed in claim 1, wherein, the process is carried out in at least one member selected from the group consisting of a stirred tank, a fixed bed or a fluidized bed.

11. The process as claimed in claim 2, wherein, the fatty compound comprises glycerides of formula: 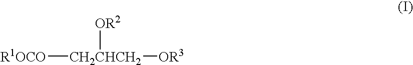
in which R¹CO is a saturated acyl group, a mono-unsaturated acyl group, or a polyunsaturated acyl group containing 2 to 22 carbon atoms and R² and R³ independently of one another represent hydrogen a mono-unsaturated acyl group or a polyunsaturated acyl group containing 2 to 22 carbon atoms.

12. The process as claimed in claim 2, wherein, the fatty compound comprises esters of monocarboxylic acids of formula:
R⁴COO—R⁵ (II)
in which R⁴CO is a saturated acyl group, a mono-unsaturated acyl group or a polyunsaturated acyl group each containing 2 to 22 carbon atoms and R⁵ is a saturated acyl group, a mono-unsaturated acyl group, a polyunsaturated acyl group, an optionally substituted alk(en)yl group containing 2 to 22 carbon atoms or the residue of a (poly)alkylene glycol.

13. A process as claimed in claim 2, wherein, the fatty compound comprises esters of dicarboxylic acids of formula:
R⁶OOC—(A)ₙ—COOR⁷ (III)
in which A is a linear or branched alkylene group containing 1 to 10 carbon atoms, an alkenylene group containing 1 to 10 carbon atoms or a phenyl group, n=0 or 1, R⁶ and R⁷ independently of one another represent a member selected from the group consisting of saturated, optionally substituted alk(en)yl groups containing 2 to 22 carbon atoms, mono-unsaturated optionally substituted alk(en)yl groups containing 2 to 22 carbon atoms, polyunsaturated optionally substituted alk(en)yl groups containing 2 to 22 carbon atoms or the residue of a polyalkylene glycol.

14. The process as claimed in claim 2, wherein, the ion exchangers comprise: anion exchangers or a combination of cation and anion exchangers arranged in series.

15. The process as claimed in claim 2, wherein, the ion exchangers comprise polymeric resins or other solid matrices containing functional groups.

16. The process as claimed in claim 15, wherein, the anion exchangers comprise at least one polymer matrix selected from the group consisting of polystyrene/divinylbenzene/dimethylamine, polystyrene/divinylbenzene/trimethyl ammonium chloride and polyacrylodimethylamine.

17. The process as claimed in claim 2, wherein, the contacting is carried out at temperatures of 0 to 150° C.

18. The process as claimed in claim 2, wherein, the process is carried out in at least one member selected from the group consisting of a stirred tank, a fixed bed or a fluidized bed.

19. The process as claimed in claim 3, wherein, the ion exchangers comprise: anion exchangers or a combination of cation and anion exchangers arranged in series.

20. The process as claimed in claim 3, wherein, the ion exchangers comprise polymeric resins or other solid matrices containing functional groups.
